(19) United States
(12) Patent Application Publication
(10) Pub. No.: US 2021/0368823 A1
(43) Pub. Date: Dec. 2, 2021

(54) METHOD OF PREPARING PREBIOTIC DIETARY FIBER AND PROBIOTIC-FIBRE COMPLEXES

(71) Applicant: Duncan-Bruce SUTHERLAND, Lonay (CH)

(72) Inventor: Duncan-Bruce SUTHERLAND, Lonay (CH)

(21) Appl. No.: 16/967,995

(22) PCT Filed: Feb. 6, 2019
(86) PCT No.: PCT/EP2019/052895

§ 371 Date: Aug. 6, 2020

(30) Foreign Application Priority Data
Feb. 6, 2018 (EP) 18155342.1

(51) Int. Cl. A23K 10/10; A23K 10/30; A23K 50/10

(52) U.S. Cl. CPC A23K 10/10; A23K 50/10; A23K 10/30

(57) ABSTRACT

A method of manufacturing prebiotic dietary fibers and probiotic encapsulated or associated material capable of affording thermal or mechanical stresses resulting from industrial feed processing as well as compositions including prebiotic dietary fibers and probiotic-fiber complexes.

### FIELD OF THE INVENTION

The invention relates to the field of prebiotic dietary fibres and probiotic integration/encapsulation, more specifically to a method of manufacturing prebiotic dietary fibres and probiotic encapsulated or associated material capable of affording thermal or mechanical stresses resulting from industrial feed processing as well as compositions comprising prebiotic dietary fibres and probiotic-fiber complexes.

### BACKGROUND OF THE INVENTION

Prebiotics are beneficial dietary fibers (or fibres) that improve the micro-biota composition and confer health advantages. Colonization of dietary plant-fibers by symbiotic microbial communities during gastro-intestinal transit supports efficient digestion of plant-based diet in a wide spectrum of vertebrates (that include humans as well as many farmed animals). The efficiency with which symbiotic intestinal microbes colonize and proliferate on plant-fibers during the gastro-intestinal transit affects the intestinal microbiota composition, nutrient availability as well as host immune-system function. Ruminants (e.g. cows) have evolved advanced biological mechanisms for digesting plant-based fibers during gastro-intestinal transit.

In animal farming, in particular, there is a need for innovations that can promote colonization of beneficial intestinal microbes on prebiotic fibers in such a way as to foster beneficial intestinal micro-biota for improved metabolic and immune-system function. E.g. weaning calves and piglets are vulnerable to infections.

Probiotics are live microorganisms that when taken as dietary adjunct confer health benefits to the recipient that include improved immune system and metabolic functions. Dense symbiotic communities of microorganisms that support digestion and immunological fitness colonize the vertebrate gut. Throughout evolution, symbiotic micro-organisms are known to co-localize within their micro-environment; for instance, in ruminants (e.g. cows), micro-organisms promoting fibre digestion in the gut including yeast, cellulolytic bacteria and archaea, adhere and co-localize on individual grass fibre strands.

For example, WO 2016/147121 A1 (ECOLE POLYTECHNIQUE FEDERALE DE LAUSANNE) discloses a food supplement comprising or consisting of Archaebacteria, and particularly methanogenic Archaebacteria, to be used as a probiotic adjunct for animal feed. The supplement can be provided to e.g. farmed animals in addition to standard feed or as a food composition. Such a supplement is particularly useful in aquaculture and proves able in increasing animal growth rates, reducing animal susceptibility to parasitic infections and/or ameliorating animal faecal waste impact on environment. Also encompassed are methods of manufacturing a composition comprising the bioactive food supplement as well as uses thereof.

U.S. Pat. No. 3,857,971 (Kamal M. Abdo et al.) discloses a feed additive for administration to ruminant animals having digestive upsets, resulting from imbalances of their rumen microbial population, which occur for various reasons, for example when the ruminants are shifted from a ration which is high in cellulose content to a ration which is high in starch content. The ruminant feed additive comprises rumen microorganisms adapted and cultured in vitro on a medium which is high in starch content. The method provides an in vitro adaptation and fermentation system for the production of selected rumen microorganisms. The aqueous suspension of adapted rumen microorganisms is mixed with an absorptive inert carrier consisting of vermiculite. In Europe as well as other geographical regions, the use of unidentified rumen microorganisms is not permitted to be registered as a feed material.

ES 2 261 059 (ALIMENTACION SIGLO XXII, S.L.) discloses cellulolytic microorganisms selected from ruminal flora and cultivated by industrial fermentation and used to provide spores for adding to food products. A method for selecting cellulolytic microorganisms from ruminal flora is also described. The selected microorganisms are cultivated by industrial-scale fermentation. Also disclosed is a food product containing spores of these microorganisms. Indeed, rumen content is acidified and incorporated in a protein-rich susbtrate and finally deshydrated to form the product of the invention.

Encapsulating probiotics with a physical barrier to improve viability in food and transit through the gastrointestinal tract has been shown to improve efficacy. Encapsulation is the process of entrapping active agents into a carrier material often with the aim to improve the incorporation into diet as well as to store them for extended periods in powder form. Encapsulation shell materials typically can include different types of polymers, carbohydrates, fats and waxes. Encapsulation aims to preserve functionality and stability of active agents throughout food processing conditions (temperature, shear force etc), drying, storage and finally through passage of the intestine. Encapsulation of probiotics for the animal feed industry is especially challenging due to the harsh extrusion processing.

Extrusion is a high temperature short time heating process to help preserve nutrient stability and facilitate digestion of starch and protein of animal feeds. For example, the temperatures reached in aquafeed extrusion are typically higher than 100 C which are generated by dissipation of mechanical energy from heated surfaces such as barrel and screw surface, shear forces between wall and material and screw and material or by injection of steam. Extruded feeds are characterized by their firm composition which is due to the gelatinization of starch.

Extrusion is the preferred industry process for aquafeed manufacture because of the higher nutrient digestibility and better stability in water. Certain dietary supplements (e.g. probiotics) lose efficacy due to heat and shear forces exerted by industry extrusion processes and, where possible, are rather subsequently added by spraying to the surface of pellets.

The viability of probiotics as well as the integrity of bioactive proteins underlies their effectiveness in food and feeds. As discussed above, industrial feed and/or food processing implies extreme temperature and shear force that reduce the viability of probiotics and the integrity of bioactive proteins.

In addition, there are currently no delivery or encapsulation systems capable of efficiently co-localizing different types of probiotics on plant fibers in the intestine and thereby facilitating symbiotic interactions between symbiotic microorganisms (e.g. yeast, bacteria, archaea).

An improved encapsulation or integration strategy is needed to ameliorate protection of probiotics and bioactive proteins against extreme temperatures (freezing and heating) as well as mechanical and chemical stresses that occur during feed processing and gastro-intestinal transit is needed. Consequently, an efficient (cost-effective and simple) way to integrate probiotic supplements into extruded feed is important because it has the potential to greatly improve the health and growth of farmed animals thus providing a feasible alternative to routine antibiotics use, which is causing a global health crisis.

It is thus an object of the invention to overcome those technical problems by designing a probiotic integration strategy able to resist to harsh feed extrusion processing so as to protect or preserve probiotic organisms.

At the same time, there is also a need to provide for a probiotic formulation able to improve nutrient absorption and immune fitness in response to diets containing fiber.

It is thus necessary to find a probiotic delivery mechanism that supports more rapid colonization of Archaebacteria associated fibrolytic microbial communities in the gut which in turn improves the efficiency of plant-digestion in the large intestine of farmed animals.

Furthermore, it is also needed to provide for a probiotic encapsulation or integration strategy that promotes colonization of fibrolytic microbiota such as archaea in the large intestine and in turn activates natural immunity corresponding to improved immune fitness.

### BRIEF DESCRIPTION OF THE INVENTION

One of the objects of the present invention is to provide a method that more efficiently associates cellulolytic microorganisms to fibres both in vitro and/or in vivo. The invention renders prebiotic dietary fibres more adherent to fiber-associated-microbes and stimulates more rapid proliferation of the fiber-associated microbiota (E.g. Mbb archaea).

In particular, one of the objects of the present invention is to provide a method of manufacturing bioactive prebiotic dietary fibrous material capable of stimulating proliferation of the fiber-associated microbiota, wherein the method is ex-vivo and comprises the steps of:
a) filtering raw rumen content through filters up to 0.20 micron so as to obtain clarified rumen fluid,
b) pasteurizing said clarified rumen fluid of step a) at a temperature of at least 50° C. for at least 1 minute so as to retain heat stable proteins,
c) adding dried cellulosic fiber material to said pasteurized clarified rumen fluid of step b) so as to form a suspension,
d) incubating said suspension at a temperature of 4-41° C. for at least 10 minutes so as to saturate the cellulosic fiber material with said pasteurized clarified rumen fluid, and subsequently collecting the resulting bioactive prebiotic fibrous material.

Also provided is a bioactive prebiotic fibrous material capable of stimulating proliferation of the fiber-associated microbiota obtained according to the ex-vivo method of any of claims 1-8, characterized in that said bioactive prebiotic fibrous material comprises rumen factors promoting adhesion and proliferation of cellulolytic-associated microbiota in vitro and in vivo and wherein said rumen factors comprise rumen proteins in the amount of at least 100 mg per liter and wherein Immunoglobulin A and/or its secretary components is present at an amount of at least 0.1 mg per gram of bioactive prebiotic fibrous material, said bioactive prebiotic fibrous material further comprising volatile fatty acids selected among acetic, propionic, valeric and butyric acids.

Further, Applicants present a probiotic entrapping or association strategy that can improve the integration of probiotics into extruded animal feed. In addition, this probiotic entrapping or integration strategy has the advantage to promote colonization of fibrolytic microbiota in the intestine by better adhering and co-localizing symbiotic microorganisms on fiber strands. The method enables for a single or combination of probiotics to be associated to the sterile bioactive prebiotic fibrous material of the invention rendering the probiotics more resilient against industrial processing due to the protective properties inherent in plant-fibers. The invention enables the probiotic cells and fiber strands to associate efficiently and form probiotic-fiber complexes characterized by novel composition and properties. The method renders the sterile bioactive prebiotic fibrous material more adherent to endogenous microbiota in the intestine of recipient animals thus facilitating (expediting) symbiotic interactions between probiotics and endogenous microbiota.

Besides, offering the advantage to promote colonization of fibrolytic microbiota in the large intestine by improving the efficiency of plant-digestion, the probiotic fiber complex composition of the invention also helps in by activating natural immunity leading to improved immune fitness.

Another object of the present invention is to provide a method of manufacturing probiotic dietary fiber complex composition capable of affording routine industrial feed or food processing comprising thermal stresses selected from −80° C. to at least 120° C. and/or mechanical stresses resulting from extruding, compacting, pelletizing, tableting, compressing or molding where the pressure or shear range is comprised between 0.1 to 700 MPa, wherein the method comprises the steps of:
a) collecting the bioactive prebiotic fibrous material of the invention,
b) mixing said bioactive prebiotic fibrous material with a fibrolytic microbiota culture in a fermentation culture to form a probiotic integrated material consisting of a probiotic fiber complex composition that is ready for industrial feed or food processing, wherein the fibrolytic microbiota culture comprises at least one population selected from Archaebacteria species.

A further object of the present invention is to provide a probiotic fiber complex composition obtained by the method according to the invention, in which said probiotic fiber complex composition contains at least 10⁶ live Archaebacteria cells per gram of said probiotic fiber complex composition and at least 0.1 mg of Immunoglobulin A and/or its secretary components per gram of said probiotic fiber complex composition. The invention also encompasses the use of the probiotic fiber complex composition or the bioactive prebiotic fibrous material in animal feed and in particular in animal farming feed or in human food.

A further object of the invention is to provide a method for increasing growth rates of animals comprising the step of providing to said animals the probiotic dietary fiber complex composition of the invention.

Other objects and advantages of the invention will become apparent to those skilled in the art from a review of the ensuing detailed description, which proceeds with reference to the following illustrative drawings, and the attendant claims.

### BRIEF DESCRIPTION OF THE DRAWINGS

FIG. 1 (a) illustrates the process summary to produce bioactive prebiotic fibrous material and probiotic-fibre complexes. Top panel, (1) dried grass fiber (Bio-Grünmehlwürfel purchased from Landi Switzerland) were integrated with (2) filtered pasteurized rumen fluid. Middle panel, the resulting product (3) bioactive prebiotic fibrous material, contains rumen factors and is more highly adhesive to (4) M. smithii probiotics than dried grass fiber alone. Bottom panel, the combination of bioactive prebiotic fibrous material with M. smithii probiotics yields (5) probiotic-fiber-complexes. The fiber forms a carrier material for the probiotics and affords protection to probiotics against heat and mechanical stresses exerted during industrial processes. Illustration adapted to represent image obtained from scanning electron microscopy (1000× objective).

FIG. 1 (b) shows the illustration of sparse low-density probiotic association to normal untreated cellulosic fiber material. Top panel, (1) dried grass fiber (Bio-Grünmehlwürfel purchased from Landi Switzerland) incubated directly with M. smithii probiotics. Bottom panel, (6) patchy low density M. smithii probiotic association to untreated fiber. Illustration representative of image as visualized under scanning electron microscope (1000× objective).

FIG. 2—Rinsing of cotton wool with PBS after cell cultivation. The optical density of PBS was measured after each rinse (3×) for each experiment (cotton wool+0 mL RF, CW+30 mL RF, CW+60 mL RF). The reference blank for the spectrophotometer analysis at 600 nm was made on PBS buffer.

FIG. 3—Cultivation of M. smithii in standard cultivation medium in presence of cotton wool subjected to different pre-treatments.

FIG. 4—Comparison of results with cotton wool soaked in different volumes (30 ml and 60 mL) of Rumen Fluid and subjected to different temperatures. The final value of optical density was obtained after 20 min of sonication.

FIG. 5—Measurement of re-solubilized crystal violet at 550 nm.

FIG. 6—Growth study of M. smithii re-cultivated from cotton wool.

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components.

As used in the specification and claims, the singular forms “a”, “an” and “the” include plural references unless the context clearly dictates otherwise.

In animal feeding, “fiber” or “fibre” is a term used to define a nutritional, not a chemical or plant anatomical concept. Fiber is the “indigestible and slowly digesting, or incompletely available, fractions of feeds that occupies space in the gastrointestinal tract” (Mertens, 1989), which defines fiber as insoluble components. Nutritionally, fiber has both physical and chemical attributes that are related to the mechanical processes of digestion (chewing and passage) and to enzymatic degradation associated with fermentation.

Ruminants such as cattle and sheep evolved as forage consumers. Plant cell walls, which we measure as fiber, cannot be digested by animals, but must be fermented by microorganisms. Fermentative digestion of fiber is slow and incomplete, and ruminants have developed many attributes that result in efficient digestion. They swallow large particles of forage and selectively retain them in the rumen to allow adequate time for fermentation. They regurgitate and rechew the large particles (rumination) to enhance digestion and allow passage through the digestive tract. During chewing, they produce salivary buffers that help maintain the pH in the rumen. Ideally, roughages could be an integral part of the diet of ruminants to take advantage of their unique digestive capability.

The terms “cellulosic fiber material” or “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

The cellulosic fiber material may be any material comprising cellulosic fibers. Examples of such materials include, but are not limited to, wood, straw, hay, grass, silage, such as cereal silage, corn silage, grass silage; bagasse, etc. A suitable material comprising cellulosic fibers is crop stover, e.g., corn stover. Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-1 18, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). In an embodiment, the cellulosic material is any biomass material. In another aspect, the cellulosic material is lignocellulose, a plant cell wall material containing lignin, cellulose, and hemicellulose in a mixed matrix. Lignocellulosic-containing material is generally found, for example, in the stems, leaves, hulls, husks, and cobs of plants or leaves, branches, and wood of trees. Lignocellulosic material can also be, but is not limited to, herbaceous material, agricultural side streams (e.g., corn stover, corn fiber, soybean stover, soybean fiber, rice straw, pine wood, wood chips, poplar, wheat straw, switchgrass, bagasse, etc.), materials traditionally used for silaging (e.g., green chopped whole corn, hay, alfalfa, etc.), forestry residues, municipal solid wastes, waste paper, and pulp and paper mill residues.

Thus in one aspect, the cellulosic fiber material is an agricultural residue. In another aspect, the cellulosic fiber material is herbaceous material (including energy crops). In another aspect, the cellulosic fiber material is municipal solid waste. In another aspect, the cellulosic fiber material is pulp and paper mill residue. In another aspect, the cellulosic fiber material is waste paper. In another aspect, the cellulosic fiber material is wood (including forestry residue).

In another aspect, the cellulosic fiber material is arundo. In another aspect, the cellulosic fiber material is bagasse. In another aspect, the cellulosic fiber material is bamboo. In another aspect, the cellulosic fiber material is corn cob. In another aspect, the cellulosic fiber material is corn fiber. In another aspect, the cellulosic fiber material is corn stover. In another aspect, the cellulosic fiber material is miscanthus. In another aspect, the cellulosic fiber material is orange peel. In another aspect, the cellulosic fiber material is rice straw. In another aspect, the cellulosic fiber material is switchgrass. In another aspect, the cellulosic fiber material is wheat straw.

In another aspect, the cellulosic fiber material is aspen. In another aspect, the cellulosic fiber material is eucalyptus. In another aspect, the cellulosic fiber material is fir. In another aspect, the cellulosic fiber material is pine. In another aspect, the cellulosic fiber material is poplar. In another aspect, the cellulosic fiber material is spruce. In another aspect, the cellulosic fiber material is willow.

In another aspect, the cellulosic fiber material is algal cellulose. In another aspect, the cellulosic fiber material is bacterial cellulose. In another aspect, the cellulosic fiber material is cotton linter. In another aspect, the cellulosic fiber material is filter paper. In another aspect, the cellulosic fiber material is microcrystalline cellulose. In another aspect, the cellulosic fiber material is phosphoric-acid treated cellulose.

In another aspect, the cellulosic fiber material is an aquatic biomass. As used herein the term “aquatic biomass” means biomass produced in an aquatic environment by a photosynthesis process. The aquatic biomass can be algae, emergent plants, floating-leaf plants, or submerged plants.

Fiber is an important and well defined component of commonly farmed animal's diets. It is known that the capacity to digest dietary fiber impacts:

How efficiently protein and energy are extracted from plant-based diets as well as the immune system function.

In animal farming improving digestion of dietary fiber is a major industry challenge since many abundant plant-based sources of protein and carbohydrate are high in fiber (e.g. algae). Currently, enzyme supplements are a favored approach to improve plant fiber digestion by farmed animals.

Fibrolytic archaea when delivered as a probiotic supplement, by forming symbiotic microbial communities, can help promote digestion of plant-based diets. Microbial communities colonize fiber in the intestine. These microbial communities that degrade plant fibers containing cellulose are referred to collectively as fibrolytic and cellulolytic microbes. Further, it is known that microbes that colonize fiber and their fermentation by-products are able to modulate immune cells since the majority of immune cells (white blood cells) in vertebrates are contained in the intestine.

The term “bioactive” refers to a compound that has an effect on a living organism, tissue/cell. In the field of nutrition bioactive compounds are distinguished from essential nutrients. While nutrients are essential to the sustainability of a body, the bioactive compounds or materials are not essential since the body can function properly without them, or because nutrients fulfil the same function. Bioactive compounds can have an influence on health.

“Prebiotics” are food ingredients that induce the growth or activity of beneficial microorganisms (e.g., bacteria and fungi). The most common example is in the gastrointestinal tract, where prebiotics can alter the composition of organisms in the gut microbiome. In diet, prebiotics are typically non-digestible fiber compounds that pass undigested through the upper part of the gastrointestinal tract and stimulate the growth or activity of advantageous bacteria that colonize the large bowel by acting as substrate for them.

As used herein, the term “probiotic” is a live microbial feed supplement which, when administered in adequate amounts, confer a health benefit on the host. The concept was introduced in the first part of the last century, by claiming that the dependence of the intestinal microbes on the food makes it possible to adopt measures to modify the flora in animal bodies and to replace the harmful microbes by useful microbes. Commonly claimed benefits of probiotics include the decrease of potentially pathogenic gastro-intestinal microorganisms, the reduction of gastro-intestinal discomfort, the strengthening of the immune system, the improvement of the skin's function, the improvement of bowel regularity, the strengthening of the resistance to cedar pollen allergens, the decrease in body pathogens, the reduction of flatulence and bloating, the protection of DNA, the protection of proteins and lipids from oxidative damage, and the maintaining of individual intestinal microbiota in subjects receiving antibiotic treatment.

A more detailed definition of probiotics relates to microorganisms that beneficially affect a host animal by modifying the host-associated or ambient microbial community, by insuring improved use of feed or by enhancing its nutrition, by enhancing the host response towards disease, or by improving quality of the ambient environment. This definition is especially appropriate when it comes to aquaculture. In fact, contrary to the terrestrial environment, where the gut represents a moist habitat in a water-limited world, in aquatic environments hosts and microorganisms share the ecosystem. Therefore, the environment for aquatic animals has much greater influence on microbiota than with terrestrials, and bacteria in aquatic medium heavily influence composition of host's gut microbiota. Aquatic animals are surrounded by an environment supporting their pathogens independently of the host animal, and opportunistic pathogens can therefore reach high densities around the fish, thus being commonly ingested with the feed or via drinking. Moreover, contrary to terrestrials which have inherent colonizing bacteria from the mother, aquatics largely spawned as axenic eggs. Ambient bacteria colonize eggs surface, and young larvae often have no developed gut (e.g., shrimp) and/or no microbial community in gut, gills or skin. As a consequence, since properties of bacteria in ambient water are very important, improvement of the ambient environment is crucial for the wellness of the bred animals.

Preferably, the probiotic used herein refers to adherent microorganisms or cells or bacteria also defined herein as adhesive microbes. Bacteria are typically found attached to and living in close association with surfaces. To effectively achieve adherence to host surfaces, many bacteria produce multiple adherence factors called adhesins. Adhesins are cell-surface components or appendages of bacteria that facilitate adhesion or adherence to other cells or to surfaces.

By “probiotic entrapment or integration” it is usually intended a process to entrap probiotics within a carrier material and it is a useful tool to improve living cells into foods, to protect, to extend their storage life or to convert them into a powder form for convenient use. In addition, integration can promote controlled release and optimize delivery to the site of action, thereby potentiating the efficacy of the respective probiotic strain. Otherwise, materials used for design of protective shell of encapsulates must be food-grade, biodegradable and able to form a barrier between the internal phase and its surroundings. Integration and in particular encapsulation is often mentioned as a way to protect bacteria against severe environmental factors. The goal of integration is to create a micro-environment in which the probiotic bacteria will survive during processing and storage and released at appropriate sites (e.g. small intestine) in the digestive tract. Thus integration such as encapsulation refers to a physicochemical or mechanical process to entrap or associate a substance in a material. In the context of the present invention, the term integration also refers to the association or entrapment of the probiotic (bacteria) in the cellulosic fiber material so as to preserve the integrity of the microorganism. Thus the term integration as used herein is to be used interchangeably with the term association. This term encompasses bacterial adhesion at all levels, starting with the initial physical attraction of bacteria to the substrate or matrix, namely the cellulosic fiber feed material. The probiotic is therefore in connection or in combination with the cellulosic fiber material so as to promote probiotic proliferation and provide a supporting structure to which probiotics can adhere. This linkage or association or integration will have the advantage of preserving the probiotic during processing and in particular during industrial feed processing.

The domain of Archaea was not recognized as a major domain of life until recently. Compared to bacteria and eukarya, their cell membrane has different biochemical components, they have a unique mechanism to generate energy using hydrogen as energy source—called methanogenesis—and so far no human pathogens has been reported, neither for animals and plants. Methanogenic archaea are known as human gut inhabitants since more than 30 years, Methanobrevibacter smithii in particular, was one of the main species that was isolated. It colonizes the gastrointestinal tract of mammals and it comprises up to 10% of all anaerobes in the colons of healthy adults. It efficiently depletes H2, reducing CO2 from the gut environment. Methanobrevibacter smithii is also the dominant methanogens also in the rumen, the first chamber of the alimentary canal of ruminant animals. Despite all the interesting features of Archaea, probiotics that are currently found in the market belongs only to other domains, i.e Eukarya (eg. yeasts) and Bacteria (eg. lactobacilli).

High cell density cultivation of Archaea is a key factor in the application of such microorganism for probiotic production. In nature, M. smithii grows in the rumen, which is rich in carbohydrate polymers (i.e fibres) indigestible to most animals and as most of microorganism, it occurs predominantly in consortia or biofilms. Surprisingly, the present invention offers a solution to develop a successful and alternative method to cultivate M. smithii on cellulosic fiber material enriched of (or supplemented with) clarified rumen fluid.

In cell culture, scaffolds are polymeric biomaterials that provide the structural support for cell attachment. The present invention is based on the finding that cellulosic fiber material such as cotton wool fibers enriched with concentrated clarified rumen fluid (see example 4) provide an ideal scaffold for the attachment of methanogenic Archaea, in this case of Methanobrevibacter smithii. This particular technique and method to cultivate in vitro M. smithii has the potential to simulate the natural growing conditions found in the gastrointestinal tract of herbivores. The end product (probiotic fiber complex composition) will result in cellulosic fiber material enriched of methanogenic Archaea that could be used as delivery system in various applications (i.e. probiotics etc. . . . ).

The study of Bang et al. (C. Bang, C. Ehlers, A. Orell, D. Prasse, S. Marlene, S. N. Gorb, S. V. Albers, and R. A. Schmitz, “Biofilm formation of mucosa-associated methanoarchaeal strains,” Front. Microbiol. vol. 5, no. JUL, pp. 1-9, 2014) shows that Methanobrevibacter smithii has the ability to grow on different surfaces and form biofilms. The strains adhered successfully to substrates such as mica and uncoated plastic dishes, forming predominantly bilayers but also multi-layered biofilms. In low amounts, it was detected a production of extracellular polysaccharides such as glucose, mannose, and galactose, which are characteristic of biofilm formation. Findings indicate also that M. smithii possess a large diversity of adhesion-like proteins (ALP), which production/regulation of those proteins seems to be specific to intestinal micro-habitats. Meaning that genes are regulated by syntrophic relationship with other members of the bacterial community, such as saccharolytic bacterias.

In particular, one of the objects of the present invention is to provide a method of manufacturing bioactive prebiotic dietary fibrous material capable of stimulating proliferation of the fiber-associated microbiota, wherein the method is ex-vivo (or in-vitro) and comprises the steps of:
a) filtering raw rumen content through filters up to 0.20 micron so as to obtain clarified rumen fluid,
b) pasteurizing said clarified rumen fluid of step a) at a temperature of at least 50° C. for at least 1 minute so as to retain heat stable proteins,
c) adding dried cellulosic fiber material to said pasteurized clarified rumen fluid of step b) so as to form a suspension,
d) incubating said suspension at a temperature of 4-41° C. for at least 10 minutes so as to saturate the cellulosic fiber material with said pasteurized clarified rumen fluid, and subsequently collecting the resulting bioactive prebiotic fibrous material.

While pasteurisation of step b) is a preferred industry standard for sterilising microbes which enables retention of functional proteins (e.g. used in dairy industry), several alternative methods exist to achieve an equivalent outcome. For example UV treatment or sterile filtration can also be used as an equivalent to pasteurization.

In the case of UV treatment it has been shown that 2 KJ/L radiation dose, treatment time of 40 see is proven to sterilise rumen fluid (highly scalable). Besides, sterile filtration with 0.2 micron pore size membrane filter would also be acceptable for smaller volumes.

However, pasteurisation is a preferred method to sterilise rumen fluid while retaining heat-stable functional proteins (E.g. IgA and sIgA) although equivalent alternative processes may be used in lieu of pasteurisation.

In a preferred embodiment of the invention, a physiologically acceptable cryoprotective reagent is added in step c) and the resulting bioactive prebiotic fibrous material of step d) is freeze-dried so as to form an inert bioactive prebiotic fibrous material.

Preferably, the physiologically acceptable cryoprotective reagent is selected among the list comprising trihalose, sucrose, ethylene glycol (EG), propylene glycol (PG; 1,2-propanediol), glycerol, formamide, butanediol (BD; 2,3-butanediol) and methylcellulose.

Spray-drying is also a commercially viable and economical preservation alternative to freeze-drying which can be performed as an alternative when the probiotic fibrous carrier material is sufficiently fine to passage through the spray-dryer nozzle without fowling.

In the context of the present invention the term “sterile” refers to a material substantially exempt of microbiological activities associated or linked to a biofilm, thus which is substantially sterile. Indeed the freeze drying step resulted in eliminating, removing, killing, or deactivating all forms of life and other biological agents (such as fungi, bacteria, viruses, spore forms, prions, unicellular eukaryotic organisms such as Plasmodium, etc.) initially present in the bioactive prebiotic fibrous material.

Alternatively it is also possible to dry the cake (rumen fluid/cellulosic fiber material) using dry air (e.g. 50° C. for 30 minutes). The drying process clearly concentrates the rumen factors to the fibres. Thus the air drying process is an alternative of freeze drying.

According to one embodiment of the invention, the filtration of step a) is performed by sequential filtering with progressively finer filtration cassettes from 10-0.20 micron filters (see example 1).

“Immunoglobulin A” (IgA, also referred to as sIgA) is an antibody that plays a crucial role in the immune function of mucous membranes. The amount of IgA produced in association with mucosal membranes is greater than all other types of antibody combined. In absolute terms, between three and five grams are secreted into the intestinal lumen each day. This represents up to 15% of total immunoglobulins produced throughout the body. IgA has two subclasses (IgA1 and IgA2) and can be produced as a monomeric as well as a dimeric form. The IgA dimeric form is the most prevalent and is also called secretory IgA (sIgA). sIgA is the main immunoglobulin found in mucous secretions, including tears, saliva, sweat, colostrum and secretions from the genitourinary tract, gastrointestinal tract, prostate and respiratory epithelium. It is also found in small amounts in blood.

The “secretory component” of sIgA protects the immunoglobulin from being degraded by proteolytic enzymes, thus sIgA can survive in the harsh gastrointestinal tract environment and provide protection against microbes that multiply in body secretions. sIgA can also inhibit inflammatory effects of other immunoglobulins. IgA is a poor activator of the complement system, and opsonises only weakly.

An advantage of obtaining IgA from a natural host such as cow (versus recombinant IgA produced with hybridomas) is that the IgA receptor repertoire is naturally diversified. The diversity of IgA receptors plays an important role in the quality and function of the IgA. This is well explained in Sutherland et al. “Fostering of advanced mutualism with gut microbiota by Immunoglobulin A” Immunological Reviews, pp 1-20; 10 Feb. 2016. Moreover, the secretory component of IgA is typically not present with recombinant IgA, only in natural derived IgA. Thus it is advantageous to obtain IgA from a natural host for the described prebiotic strategy of the invention.

Surprisingly Applicant has found that when heating the rumen content it helps making the rumen fluid less viscous and passes more readily through the filters. These proteins are a bit like adhesive glue binding microbe to fiber—thus heating the rumen content will help to extract/dissociate IgA/sIgA. Thus this heating treatment of the rumen represents an important step to dissociate IgA/sIgA and other adhesive proteins from the fiber into the rumen fluid.

In accordance with the present invention, the dried cellulosic fiber material of step c) is selected among algae; plants such as forage including lignin, cellulose and structural carbohydrates such as fibrous cellulose and hemi-cellulose.

In one preferred embodiment of the invention, the weight ratio of the pasteurized clarified rumen fluid to the dried cellulosic fiber material in step c) is 5:1. However, according to another embodiment, the weight ratio of the pasteurized clarified rumen fluid to the dried cellulosic fiber material in step c) is 1:1.

In one embodiment of the invention, the incubation of step d) is performed under agitation. Agitation may consist of circular rotations or rocking movements as is commonplace. Circular rotation may typically be automated to 120 rpm however other rotation speeds can be contemplated by the skilled in the art.

Also provided is a bioactive prebiotic fibrous material capable of stimulating proliferation of the fiber-associated microbiota, obtained according to the method of the invention and wherein said bioactive prebiotic fibrous material comprises rumen factors promoting adhesion and proliferation of cellulolytic-associated microbiota in vitro and in vivo and wherein said rumen factors comprise rumen proteins in the amount of at least 100 mg per liter and wherein Immunoglobulin A and/or its secretary components is present at an amount of at least 0.1 mg per gram of bioactive prebiotic fibrous material, said bioactive prebiotic fibrous material further comprising volatile fatty acids selected among acetic, propionic, valeric and butyric acids.

In particular, the bioactive prebiotic fibrous material of the invention contains rumen factors originated from rumen fluid contains heat-stable rumen proteins, notably secretory IgA as well as short-chain-fatty acids (also called volatile fatty acids). Volatile fatty acids include acetic, propionic, butyric, valeric, as well as isobutiric and isovaleric acids.

Interestingly, the bioactive prebiotic fibrous material of the invention comprises Immunoglobulin A (IgA) and/or its secretary components at a concentration of at least 0.1 mg (100 micrograms) per gram of bioactive prebiotic fibrous material. While this is similar to physiological IgA level in saliva and gastrointestinal (GI) tract, the IgA and sIgA contained in the resulting bioactive prebiotic fibrous material is not the same as found in nature (since it has been pasteurized and is devoid of the rumen microbes).

As disclosed in Mach et al. “Secretory IgA, A major Immunoglobulin in Most Bovine External Secretions”, J. Immunol.; Vol. 106, pp 552-563; No. 2, February 1971, the physiological concentration of IgA and sIgA in the saliva and rumen fluid of a cow can amount to 1 mg IgA/100 mL rumen fluid or saliva. In case one uses 10 mL rumen fluid/gram of dried cellulosic fiber material, the concentration of IgA and sIgA amounts to 0.1 mg/gram of the resulting bioactive prebiotic fibrous material.

Preferably the concentration of IgA and sIgA amounts to 0.2 mg/gram, more preferably to 0.5 mg/g, even more preferably to 0.8 mg/gram and most preferably to 1 mg/gram of the bioactive prebiotic fibrous material.

Associating or integrating the filtered pasteurized rumen extract with fibrous feed material by incubation and cryo-preservation forms a new stable product characterized by (a) composition and (b) functional properties. The composition and function of the bioactive prebiotic fibrous material of the invention can be summarized as follows:
a) The composition is characteristic in that it contains ruminant proteins (resistant to 65° C.), more specifically immunoglobulin (including the secretory component of IgA) adhered to the bioactive prebiotic fibrous material.
b) The function is characterized by improved adherence of the prebiotic fibrous feed material to cellulolytic-associated-microbiota both in vitro and in vivo.
c) The function is characterized in that the bioactive prebiotic fibrous material can catalyse proliferation of cellulolytic-associated-microbiota both in vitro and in vivo.
d) The function is characterized in that the plant-fibres (bioactive prebiotic fibrous material) once associated to probiotics can afford a protective shield against industrial processing (heat and shear force).

It is another object of the present invention to provide a method of manufacturing probiotic dietary fiber complex composition capable of affording routine industrial feed or food processing comprising thermal stresses selected from −80° C. to at least 120° C. and/or mechanical stresses resulting from extruding, compacting, pelletizing, tableting, compressing or molding where the pressure range is comprised between 0.1 to 700 MPa, wherein the method comprises the steps of:
a) collecting the bioactive prebiotic fibrous material of the invention,
b) mixing said bioactive prebiotic fibrous material with a fibrolytic microbiota culture in a fermentation culture to form a probiotic integrated material consisting of a probiotic fiber complex composition that is ready for industrial feed or food processing, wherein the fibrolytic microbiota culture comprises at least one population selected from Archaebacteria species such as methanobrevibacter (Mbb) species and preferably Methanobrevibacter smithii.

“Industrial feed processing” refers to physical or chemical changes in feedstuffs, which influence their nutritional value. Industrial feed processing usually implies “Thermal or mechanical stresses” to probiotic cells or bacteria.

Among others, mechanical stresses can be selected among extruding, compacting, pelletizing, tableting, compressing or molding whereas thermal stresses are selected among cryogenization, cryopreservation, freezing, freeze drying, heating or the like.

According to a particular embodiment of the invention, “industrial food processing” is the transformation of raw ingredients, by physical or chemical means into food, or of food into other forms. Food processing combines raw food ingredients to produce marketable food products that can be easily prepared and served by the consumer. Food processing typically involves activities such as mincing and macerating, liquefaction, emulsification, and cooking (such as boiling, broiling, frying, or grilling); pickling, pasteurization, and many other kinds of preservation; and canning or other packaging. Primary-processing such as dicing or slicing, freezing or drying when leading to secondary products are also included.

Ruminants, such as cattle, rely upon a rich and diverse community of symbiotic ruminal microbes to digest their feed. These symbionts are capable of fermenting host-indigestible feed into nutrient sources usable by the host, such as volatile fatty acids. Rumen microbes can be assigned to different functional groups, such as cellulolytics, amylolytics, proteolytics, etc., which degrade the wide variety of feed components or further metabolize some of the products formed by other microbes.

“Fibrolytic microbiota” also referred herein as “Fiber-associated microbiota” especially “Rumen microbiota” consist in a symbiotic community of anaerobic microorganisms containing bacteria, protozoa, fungi and archaea that collectively degrade cellulose. The rumen microbiota is characterized by methanobrevibacter (Mbb) species that metabolize hydrogen by-products resulting from cellulose fermentation into methane. The terms rumen microbiota both refer to microbiota extracted from the rumen of a ruminant as well as artificial rumen microbiota prepared in laboratory by a skilled in the art.

Preferably, the bioactive prebiotic fibrous material of step a) is selected among algae; plants such as forage including lignin, cellulose and structural carbohydrates such as fibrous cellulose and hemi-cellulose.

More preferably, the bioactive prebiotic fibrous material of step a) is sterile so as to avoid the addition of any undesired contaminants.

In general, the duration of the fermentation of step b) will be decided taking into account that incubation should be continued for a sufficient length of time to ensure satisfactory attachment/association or integration of the probiotic cells present in the fibrolytic microbiota culture to the bioactive prebiotic fibrous material so as to form the probiotic fiber complex composition of the invention.

The temperature of the fermentation of step b) should be selected taking into account the particular requirements of the fibrolytic microbiota culture used according to the invention. Usually the temperature is selected in the range of 10° C. to 60° C., e.g., in the range of 15° C. to 50° C., in the range of 20° C. to 45° C., in the range of 25° C. to 40° C., in particular about 37° C. and preferably about 38.5° C.

According to a particular embodiment of the invention, the resulting probiotic (dietary) fiber complex composition is in a solid form.

In accordance with a preferred embodiment of the invention, the probiotic to be integrated or associated following the method described above is a fibrolytic or cellulolytic microbe such as Archaebacteria species.

“Fibrolytic microbe or cellulolytic microbe” constitute a group of microorganisms that are able to process complex plant polysaccharides thanks to their capacity to synthesize cellulolytic and hemicellulolytic enzymes. Polysaccharides are present in plant cellular cell walls in a compact fiber form where they are mainly composed of cellulose and hemicellulose. Fibrolytic enzymes, which are classified as cellulases, can hydrolyze the β (1->4) bonds in plant polysaccharides. Cellulase and hemicellulase (also known as xylanase) are the two main representatives of these enzymes.

According to a preferred embodiment of the invention, the supplemented fibrolytic microbiota culture of step b) contains yeast, cells, microbes and/or extracts or fragments thereof and at least one population selected from Archaebacteria species such as methanobrevibacter (Mbb) species and preferably Methanobrevibacter smithii.

Ideally, the supplementation of fibrolytic microbiota culture in step b) is performed by adding between 10⁶ to 10¹² fibrolytic microbiota cells per gram of bioactive prebiotic fibrous material.

More preferably, the fibrolytic microbiota culture or probiotic comprises at least one population selected from Archaebacteria species also referred herein as methanogenic Archaebacteria species.

The term “population” as used herein relates to a group of individual organisms of the same species defined by time and space. However, the term can be also intended as a community, i.e. a group of organisms inhabiting a particular ecological niche, which could include any number of species. In this context, the term “population” is also referred to as a “mixed population”.

A list of “methanogenic Archaebacteria species” suitable in carrying out the invention comprises Methanobacterium bryantii, Methanobacterium formicuin, Methanobrevibacter arboriphilicus, Methanobrevibacter gottschalkii, Methanobrevibacter ruininantiuin, Methanobrevibacter smithii, Methanococcus chunghsingensis, Methanococcus burtonii, Methanococcus aeolicus, Methanococcus deltae, Methanococcus jannaschii, Methanococcus maripaludis, Methanococcus vannielii, Methanocorpusculum labreanum, fethanoculleus bourgensis, Methanoculleus marisnigri, Methanoflorens stordalenmirensis, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophila, Methanosarcina acetivorans, Methanosarcina barkeri, Methanosarcina mazei, Methanosphaera stadtmanae, Methanospirillium hungatei, Methanothermobacter defluvii, Methanothermobacter thermautotrophicus, Methanothermobacter thermoflexus, Methanothermobacter wolfei and Methanothrix sochngenii. In one preferred embodiment, the Archeabacteria species is a methanobrevibacter (Mbb). Even more preferably, the Methanobrevibacter species used as active agent for the probiotic fiber complex composition of the invention is the Methanobrevibacter smithii species.

In this preferred embodiment of the invention, the active agent of the methanobrevibacter (Mbb) fiber complex composition is a member of the methanogenic Archaebacteria species, that is, Archaebacteria species that produce methane as a metabolic by-product in anoxic conditions. Methanogens are a diverse group of strict anaerobes which are widely distributed in nature and can be found in a variety of permanently anoxic habitats like flooded soils, sediments, sewage-sludge digesters or the digestive tract of certain animals. All known methanogens are affiliated to the Archaea and extremely sensitive to oxygen. The hallmark feature of methanogens is the reduction of C-1 compounds (e. g., CO₂, methanol, formate, or N-methyl groups) to methane (CH₄). Methanogens play a vital ecological role in anaerobic environments of removing excess hydrogen and fermentation products that have been produced by other forms of anaerobic respiration. Methanogenic Archaea also play a pivotal role in ecosystems with organisms that derive energy from oxidation of methane, many of which are bacteria, as they are often a major source of methane in such environments and can play a role as primary producers. Methanogens also exert a critical role in the carbon cycle, breaking down organic carbon into methane, which is also a major greenhouse gas. Methanogenesis also occurs in the guts of humans and other animals, especially ruminants. In the rumen, anaerobic organisms, including methanogens, digest cellulose into forms usable by the animal. Without these microorganisms, animals such as cattle would not be able to consume grass. The useful products of methanogenesis are absorbed by the gut, while methane is released by the animal.

Without being bound to theory, the supplemented fibrolytic microbiota culture of adherent probiotic of step b), preferably methanobrevibacter (Mbb) strains (e.g. Mbb smithii) will bind or glue to the sterile bioactive prebiotic fibrous material also defined as fibrous scaffold or matrix and will colonize it to form a probiotic integrated or associated material corresponding to a probiotic fiber complex composition, preferably a Mbb-fibre complex composition that is ready for industrial feed processing.

Applicants have surprisingly observed that the probiotic fiber complex composition of the invention results in improved cryopreservation/thermostability/mechanical resistance and better viability when freeze-dried and extruded than when probiotic is processed alone or using conventional carriers. Thus the method of the invention leads to the improvement of probiotic resistance to processing stresses and their effectiveness in recipient animals.

It is another object of the invention to provide a probiotic fiber complex composition obtained by the method of the invention, wherein said probiotic fiber complex composition contains at least 10⁶ live Archaebacteria cells per gram of said probiotic fiber complex composition and at least 0.1 mg of Immunoglobulin A and/or its secretary components per gram of said probiotic fiber complex composition.

In particular, the probiotic fiber complex composition contains from 10⁶-10¹² live Archaebacteria cells per gram of said probiotic fiber complex composition.

In accordance with the invention, the probiotic fiber complex composition is a Archaebacteria fiber complex composition, preferably a methanogenic Archeabacteria fiber complex composition and even more preferably a methanobrevibacter fiber complex composition (Mbb) such as Methanobrevibacter smithii fiber complex composition.

The vertebrate gut is colonized by dense symbiotic communities of micro-organisms that support digestion and immunological fitness for their host. Throughout evolution, symbiotic micro-organisms are known to co-localize within their micro-environment; for instance, in ruminants (e.g. cows), micro-organisms promoting fiber digestion in the gut including yeast, cellulolytic bacteria and archaea, adhere and co-localize on individual grass fiber strands.

Applicant has modelled a novel prebiotic fibrous material namely a carrier system that mimics ‘nature's design’ by associating probiotic microorganisms onto plant fiber strands. Probiotics are beneficial live microbial strains, encapsulated and ingested by humans and animals to support health. In agriculture, probiotics, by promoting digestion, improve the feed conversion ratio of farm animals and simultaneously act as immune stimulants.

Thus advantageously the invention helps to:
promote symbiotic interactions between multiple probiotic strains

Harnessing plant fiber as carrier material for probiotics has other potential benefits. The evolution of fiber matrices provides plants and grass with structurally protective properties that help to preserve plant cells against mechanical, thermal and chemical exposures encountered in their habitats. By using plant fibers as carrier material for probiotics there is also potential to:
shield probiotics against mechanical, thermo and chemical damage

The probiotic fiber complex composition of the invention also referred as multi-probiotic-carrier system supports synergy between multiple probiotic combinations as well as boosts probiotic viability when incorporating into food and farm feed. Such an invention has excellent potential to improve antibiotics-free farming around the world.

As will be apparent to a person skilled in the relevant art, a population of methanobrevibacter (Mbb) species for inclusion into the methanobrevibacter (Mbb) fiber complex composition can be obtained, if a commercially-available alternative is not envisaged, through any common isolation method, including the serial dilution method, streak plate method, pour plate/spread plate method, enrichment culture method, methods exploiting selective media, methods exploiting differential media and so forth.

In particular, the methanobrevibacter (Mbb) fiber complex composition of the invention is characterized by the fact of comprising at least one population of at least one Methanobrevibacter species. However, several other agents can be present in the supplement, particularly other kind of probiotics. This is especially true when, as will be detailed later on, said Methanobrevibacter population has been obtained from catties' rumen extracts, where a blend of several microorganisms (generally named microbiota) can be present. Without being necessarily bound to this theory, some observations made by the present inventors suggest that Archaebacteria populations maintain a positive symbiotic relationship and promote suitable environmental growth/proliferation conditions of a so called “Archaea associated microbiota” (i.e. an ensemble of microorganisms that usually establish a symbiotic tie in a selected environment with Archaeabacteria, including for example anaerobic/fermenting probiotics), particularly in terms of preservation of a complex population comprising more types of anaerobic microorganisms. An equilibrium between more than one probiotic in a food supplement enriched in Archaebacteria according to the invention is possibly one of the key features of the noticed positive effects of the supplement of the invention on farmed animals.

As would be also evident for a skilled person, a population of at least one Archaebacteria such as methanobrevibacter species can be obtained with any known method, such as purchase on the trade of isolated Archaebacteria strains (including lyophilized forms thereof), culture of Archaebacteria in suitable culture broths (such as for instance, the Methanosphaera Medium I or the Methanobacterium Medium from the Leibniz Institute DSMZ—German Collection of Microorganisms and Cell Cultures GmbH) with or without a pelleting step, and the like.

The rumen extract represents a perfect culture medium adjunct for Archaebacteria since it contains nutrients that nourish the microorganisms (especially methanogenic Archaea) under perfect culture conditions—anaerobic conditions in the cow rumen —. Through routine lab procedures, large amounts of rumen extract can be extracted from one cow per day; this can be possibly sterilized (through e.g. exposure to oxygen and/or extreme temperatures) and a rumen fluid obtained therefrom can be used as the basis to cultivate Archaebacteria under anaerobic, controlled lab conditions.

The invention also encompasses the use of the probiotic fiber complex composition or the bioactive prebiotic fibrous material of the invention in animal feed and in particular in animal farming feed or in human food.

As described herein, the probiotic fiber complex composition of the invention and preferably the methanobrevibacter (Mbb) fiber complex composition, is intended for use in animal feed and in advantageously in animal farming feed.

In a preferred embodiment of the invention, the farmed animals referred to in the present invention are birds, mammals or aquatic animals (such as shrimps, crustaceans or fishes). The invention is however not limited to this use. The feed/food supplement may also be administrated to pets, captive animals or human beings.

Thus according to a specific embodiment, the probiotic fiber complex composition of the invention is for use in human food such as for example a food supplement or a food additive.

It is a further object of the invention to provide a method for increasing growth rates of animals comprising the step of providing to said animals the probiotic fiber complex composition and preferably the methanobrevibacter (Mbb) fiber complex composition of the invention.

It is yet a further object of the invention to provide a method for ameliorating an animal fecal waste impact on environment comprising the step of providing to said animal the probiotic fiber complex composition and preferably the methanobrevibacter (Mbb) fiber complex composition of the invention.

Another object of the invention is to provide the probiotic fiber complex composition and preferably the methanobrevibacter (Mbb) fiber complex composition, for use in a method for reducing susceptibility to parasitic infections of animals.

One aim of the probiotic fiber complex composition and in particular the methanobrevibacter (Mbb) fiber complex composition of the invention is boosting and/or enhancing certain aspects of the physiology of farmed animals, as well as the consequent impact said ameliorated physiology-related conditions have on the surrounding environment. As explained above, this is especially true in the aquaculture, where the farmed animals (in this case, aquatic animals such as for example fishes, eels or crustaceans) have an extremely tight relationship with the environment they are farmed in. However, farmed animals according to the invention can also be birds such as chickens, fowls, ostriches and the like, or mammals as for example domesticated animals such as cattle, sheep, pigs, horses, rodents and the like, and also primates and humans. Accordingly, the probiotic fiber complex composition i.e. the methanobrevibacter (Mbb) fiber complex composition is characterised by the fact that it acts on physiological animal parameters, by positively affecting them so that farming conditions are advantageously improved compared to standard farming conditions. In particular, the methanobrevibacter (Mbb) fiber complex composition of the invention results useful in increasing animal growth rates and/or in reducing animal susceptibility to parasitic infections and/or in ameliorating the animal faecal waste impact on environment.

According to an embodiment, it has been shown that the methods of the present invention increase the digestibility of a cellulosic material by at least 5%, e.g., at least 100%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, up to 100%. Increased digestibility of cellulosic material is measured pursuant to in vitro true digestibility (IVTD) procedures.

IVTD is an anaerobic fermentation performed in the laboratory to simulate digestion as it occurs in the rumen. Rumen fluid is collected from ruminally cannulated high producing dairy cows consuming a typical total mixed ration (TMR). Forage samples are incubated in rumen fluid and buffer for a specified time period at 39° C. (body temperature). During this time, the microbial population in the rumen fluid digests the sample as would occur in the rumen. Upon completion, the samples are extracted in neutral detergent solution to leave behind the undigested fibrous residue. The result is a measure of digestibility that can be used to estimate the digestibility of cellulosic materials; e.g., corn stover, corn fiber, soybean stover, soybean fiber, rice straw, pine wood, wood chips, poplar, wheat straw, switchgrass, bagasse, etc. In general, the higher the value of IVTD, the higher is the digestibility of the forage and the higher is the feed value of the forages for feeding ruminants.

It has been surprisingly observed that probiotic fiber complex composition and preferably methanobrevibacter (Mbb) fiber complex composition of the invention is able to:
(i) improve energy harness leading to better growth rates,
(ii) increase resistance to infections by reducing the need for broad-spectrum antibiotic treatments and
(iii) improve quality of discharges resulting in less impact on the environment.

For example, the Mbb-fibre complex of the invention results in better probiotic functionality and potency than when Mbb or fibre are administered to animals independently.

In addition, the probiotic fiber complex composition i.e. the Mbb-fibre complex of the invention when administered to the diet promotes fibrolytic digestion and promotes adaptation to herbivory.

It has also been shown that Mbb-fibre complexes of the invention administered to the diet result in better FCR (feed conversion ratio), feed digestibility and growth rates in animals than when Mbb and fibre are administered to the diet independently.

Therefore the potency is higher at lower doses making the probiotic more cost-effective.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

Preparation of Sterile Bioactive Prebiotic Fibrous Material:

Step 1. Filtration of Rumen Fluid to Obtain Clarified Rumen Fluid

Collect 110 Kg raw rumen content. Warm to 50° C. and mix vigorously to dissociate proteins into the liquid fraction. Place rumen contents (solid and liquid materials) in nylon fabric filter with 1 ml holes, centrifuge 300 rpm for 10 minutes to obtain 35 L of rumen fluid (RF).

Next, continue to perform sequential filtering with progressively finer filtration cassettes as follows:

3.0 micron filter

0.9 micron filter

0.25 micron filter

0.22 micron filter

The sequential filtration process (starting with 110 Kg raw content) will yield in the order of 10 L of clarified rumen fluid.

The clarified rumen fluid may be stored by refrigeration or freezing.

Step 2. Pasteurization of Clarified Rumen Fluid

Pasteurization (50° C.-70° C.) further ensures the inactivation of microorganisms including viruses and denatures the majority of proteins leaving in fact only heat stable proteins. Pasteurization at 65° C. for 15 minutes has been demonstrated to retain secretory Immunoglobulin-A and is a preferred pasteurization method (Kinetic and Thermodynamic Parameters for Heat Denaturation of Bovine Milk IgG, IgA and IgM G. MAINER, L. SA' NCHEZ, J. M. ENA, and M. CALVO. Volume 62, No. 5, 1997-JOURNAL OF FOOD SCIENCE-1035).

Total concentration of protein in purified rumen fluid was determined by the Bicinchoninic acid (BCA) assay kit (ThermoFisher Scientific, Switzerland). Additionally, the BCA compatable kit (ThermoFisher Scientific, Switzerland) was also used to previously eliminate any possible interferences to the BCA assay. Purified RF samples were diluted 2 fold in PBS. Bovine serum albumin (BSA) was used as the protein standard for the calibration curve. To diluted standards of BSA and RF were added reagents A and B of BCA compatible kit, according to the manufacturer's protocol, to precipitate the protein. Subsequently, reagents A and B of BCA assay kit were mixed in proportions 50:1 (A:B) according to manufacturer's instructions and incubated for 30 min at 37° C. The Cu²⁺ ions present in the reagents complex with certain amino acids of the proteins, giving a characteristic purple color. The concentration of protein is therefore proportional to the absorbance (560 nm) of the final complex. This assay has been proven useful to characterize the total amount of protein of purified RF and track any possible quality changes from batch to batch. The concentration of proteins in Rumen Fluid showed some variation falling within the range of 0.5-1 mg per mL depending on batch and season.

Bovine sIgA is homologous to human sIgA and common antigenicity of bovine and human IgA has previously been demonstrated (Mach et al, 1971). Bovine sIgA concentration was measured by ELISA method. sIgA concentration in rumen gastro intestinal secretions typically exceeds 0.1 mg per mL.

Step 3. Integrating Rumen-Fluid-Extract with Fibrous Feed Material

Cryoprotective reagent (E.g. trihalose) is dissolved in the rumen-fluid-extract from step-2. Next, dried fibrous feed material (E.g. dry grass feed) is immersed in rumen-fluid-extract from step-2 containing the cryoprotective reagent. The preferred dry-wet weight ratio is 1:1. The suspension is incubated at physiological temperature 37-38° C. for 1-6 hours in order to saturate the feed material with the rumen fluid and also to enable molecular bonding. The saturated feed material is preserved and stabilized employing routine freeze-drying procedure to form an end product comprising fibrous feed integrated with the selected rumen fluid factors. The fibrous feed material once integrated with the selected rumen fluid factors has two key advantages:
is more adherent to cellulolytic-associated-microbiota
is effective at promoting growth of cellulytic-associated microbiota

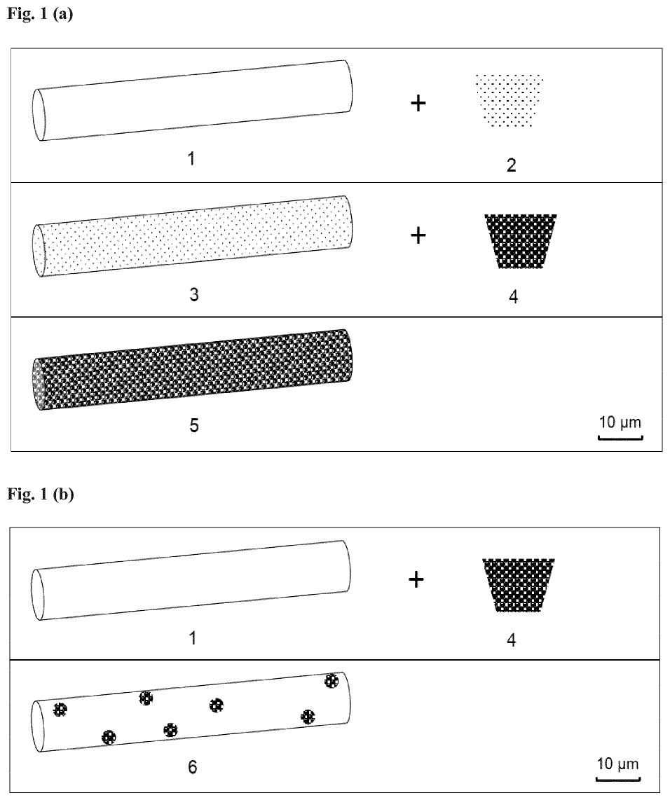

Scanning electron microscopy reveals that fibrous feed material integrated with the selected rumen factors readily adheres M. smithii cells as depicted in FIG. 1a bottom panel. In contrast, scanning electron microscopy shows that normal untreated rumen fibrous feed material does not as readily adhere to M. smithii cells (FIG. 1b).

When the fibrous feed material integrated with rumen factors is mixed into M. smithii culture, the growth rates of M. smithii are significantly increased and reach higher densities than cultures enriched either with rumen fluid or fiber material alone. Thus the enrichment of rumen factors in fiber material is responsible for the increased proliferation of M. smithii in vitro.

TABLE 1
Difference between initial and final OD 600 nm in experiment
with different concentration of prebiotic

| Prebiotic amount | ODᵢₙᵢ | OD_{fin} | ΔOD |
|---|---|---|---|
| 0.0 g/20 mL | 0.294 | 0.45 | 0.156 |
| 0.1 g/20 mL | 1.935 | 4.55 | 2.615 |
| 0.2 g/20 mL | 3.26 | 7.55 | 4.29 |
| 0.5 g/20 mL | 7.325 | 15.3 | 7.975 |

Note about controls: Supplementation of the growth media with 10% clarified rumen fluid alone resulted in maximum ΔO.D. of 0.665. In contrast the prebiotic contains an equivalent of only 2.5% rumen fluid but yields ΔO.D. of 7.975. Normal fibers (not integrated with RF) when added to culture media yielded ΔO.D 0.2. Thus the novel bioactive prebiotic fiber integrated with rumen factors has a potent effect on M. smithii proliferation in vitro.

### Example 2

Preparation of Probiotic-Fiber-Complex Composition Capable of Affording Thermal or Mechanical Stresses Resulting from Industrial Feed or Food Processing

The sterile bioactive prebiotic fiber material prepared as in example-1 is added at a ratio of 1:10 to M. smithii standard growth media containing 10¹² M. smithii cells per mL media and incubated anaerobically gassed with 4 parts H₂ and 1 part CO₂, under 2 bars pressure for 6 hours as is routine for M. smithii culture. The fiber material is afterwards isolated/separated from the culture media using a nylon mesh strainer (40 micron). Scanning electron microcoscopy reveals the fiber material is densely colonized with/adhered to by M. smithii cells. The product is defined as probiotic-fiber-complex. The probiotic-fiber-complex composition may be preserved using such standard cryopreservation methods as freeze-drying.

Scanning electron microscopy reveals that standard fibers (not integrated with pasteurized clarified rumen fluid) are not readily colonized by M. smithii. The adhesion of M. smithii is supported by the integration of the said rumen factors within the fiber.

M. smithii probiotic-fiber-complex composition of the invention when heated to 75° C. for 5 minutes remain viable since they are capable of proliferating efficiently following the heat pulse when incubated in standard M. smithii culture conditions. In contrast, M. smithii cells suspended freely in culture media without association to fiber carrier material once heated to 75° C. for 5 minutes no longer remain viable following heat pulse since they do not proliferate in standard M. smithii culture conditions. The probiotic-fiber-complex is thus afforded protection to heat treatment.

No comparative test is possible since the density of M. smithii on conventional fibres is too low. If the cell density is too low it is very difficult for the cell proliferation to occur in media in the first place (either takes extremely long to start proliferating or doesn't proliferate at all—a minimum biomass is needed for M. smithii culture to be effective). The fact that M. smithii does not adhere well to normal fibres (not treated according to the method of the present invention) implies that they cannot be good carriers of M. smithii (ie. the density of cells is too low to use for any meaningful application in vitro or similarly in vivo).

Thus even without the heat treatment the low density of M. smithii on normal fibres are not likely to supply sufficient density of M. smithii to inoculate cultures in an efficient way.

### Example 3

Processing of Rumen Content and Porphyra Seaweed to Form a Cellulosic Fiber Feed Material as Probiotic Carrier:

Rumen content (RC) containing partially digested forage was harvested from healthy organic cows (Uelihoff abattoir Ebikon, Switzerland) following slaughter. RC was heated to 50° C. and mixed vigorously for 15 minutes and/or sonicated to dissociate proteins from the solid fraction into the liquid fraction. Heating and agitating the rumen content increases the protein concentration in the rumen fluid fraction. The treated RC was then pressed using a grape-press to collect the rumen fluid fraction while the solid fraction was discarded. The warm rumen fluid fraction was filtered with progressively finer filtration cassettes as follows:

3.0 micron filter

0.9 micron filter

0.25 micron filter

0.22 micron filter

The rumen fluid passes more readily through the filters (less filter fowling) when it is heated. The clarified rumen fluid was further pasteurized at 55° C. for 15 minutes.

Formation of the Probiotic-Fibre Complex Composition:

Dry shredded porphyra seaweed (red algae) obtained from Fukuoka Ariake Japan Fisheries was sterilized at 65° C. in Schott bottles and then incubated with the clarified pasteurized rumen fluid preparation at a ratio of 1:5 (porphyra to rumen fluid) at 38° C. with gentle agitation for 2 hours to allow association of rumen proteins (sIgA) to the porphyra seaweed (fibrous material). The porphyra-rumen fluid mix was next transferred into an anaerobic fermenter containing methanobrevibacter growth media DSMZ-119a (90 L) and Methanobrevibacter smithii (Mbb) and was cultured for 5 days obtaining usual density of 10¹² Mbb smithii cells per liter of growth media (as determined by U.V. fluoro-microscopic analysis in a cell counting chamber). The presence of treated porphyra in the growth media did not prevent the growth of Mbb smithii cultures in the anaerobic fermenter. The fibrous porphyra was filtered and separated from the growth media and next preserved by freeze-drying Telstar Lyobeta with addition of cryo-protectant agent trehalose. Microscopic analysis (using scanning electron microscope, EOL JSM-6510LV at 1000×) magnification revealed adherence of dense groups of Mbb to the treated porphyra fibres. The addition of treated phorphyra (integrated with rumen fluid proteins) to the Mbb smithii anaerobic culture resulted in formation of Mbb-fibre complexes. In contrast Mbb does not readily colonize untreated porphyra indicating that rumen fluid proteins play an important role in the association of microbes to the plant-fibrous material.

Probiotic-Fibre Complex Composition is Resistant Against Forces Exerted During Aquaculture Feed Extrusion Processing:

In this example 5 Kg of the obtained Mbb-fibre complex composition was mixed with 95 Kg shrimp fish-feed-mix (containing as follows) prior to extrusion and pelleting (non-complexed Mbb and untreated porphyra were used as a control).

Ingredient composition %

| | |
|---|---|
| Soybean meal, 48% CP | 30 |
| Wheat flour | 24 |
| Wheat | 16.23 |
| Fish meal | 10 |
| Soy protein concentrate | 7.85 |
| Fish oil | 2.5 |
| PRODUCT | — |
| Premix Min & vitamin | 2 |
| Lecithin | 2 |
| Squid meal | 2 |
| Dicalcium phosphate 22 | 1.5 |
| Soybean oil | 1 |
| MetAMINO | 0.35 |
| ThreAMINO | 0.25 |
| L-histidin | 0.17 |
| AQUAVILys | 0.15 |
| Crude Protein | 34.37 |
| Crude Fat | 8.47 |
| Starch | 24.25 |
| Gross energy | 18 |
| Crude fibre | 2.54 |
| Crude Ash | 6.03 |
| P | 0.89 |
| DHA | 0.05 |
| Lys | 2 |
| Met | 0.92 |
| Met + Cys | 1.4 |
| thr | 1.5 |
| Trp | 0.4 |
| Arg | 2.21 |
| Ile | 1.4 |
| Leu | 2.44 |
| Val | 1.53 |
| His | 1 |
| Phe | 1.56 |

During “proof of concept” evaluations, extrusions were performed using a pilot-scale, co-rotating, intermeshing, twin-screw extruder (DNDL-44, Buhler AG, Uzwil, Switzerland) with a smooth barrel and a length/diameter ratio of 32:1 (1422 mm long and 44 mm screws). The barrel of the extruder consists of 6 temperature-controlled sections. Sections 2, 3, 4, and 5 are heated by steam and section 6 is digitally controlled by heated recirculating water (model HY 4003HP, Mokon, Buffalo, N.Y.). The screws are built to have a feed section, mix section, a work section with reversed screw elements, and a final conveying section. The screw speed was 400 rpm. The barrel temperatures were set between ambient and 70° C. The aqua feed was extruded through a die with 90 1.0 mm diameter holes and cut into short lengths with a rotating face cutter. The resulting pellets had a size of 1.0 mm.

The survival of probiotics in extruded aqua feed was evaluated by homogenizing the aqua feed pellets in growth media (methanobrevicater media DSMZ 119a) and then applying the appropriate fermentation conditions for the Mbb smithii probiotic microbes (37° C., 2 bars pressure, 4:1 ratio of H2 and CO2 gases for 96 hours) in a Ralf 6.7 L BioEngineering (Wald, Switzerland) fermenter. Mbb protected in the fibre complex composition that had been extruded were able to reactivate and multiply in fermenters much more efficiently (as determined by optical density, fluoro-microsopic enumeration and PCR) indicating that probiotic-fibre complex formation preserved viability of Mbb smithii during aqua feed extrusion processing. In contrast when the probiotic Mbb was extruded without first being integrated to fibre material, the growth rate of the probiotic was considerably lower compared to when the probiotic Mbb was integrated in the prebiotic fibrous material of the invention (even if the same amount of fibre material was present in the feed mash). Thus the direct attachment of Mbb to the fibrous material treated according to the method of the invention (facilitated by rumen proteins) supports cell viability during feed extrusion. However when Probiotic Mbb was extruded in the presence of the same amount of fibre material in the feed mash cell but without any treatment viability was much lower compared to Mbb mixed with the prebiotic fibrous material of the invention.

Improved Efficacy of Probiotics Following Encapsulation with Fibre Complexes.

Extruded shrimp feed containing either
1. Mbb-fibre complexes, diet-1
2. Mbb and fibre added separately (not complexed), diet-2 (experimental control) were fed to white leg shrimp for feeding trials and for acute stress challenges and immune parameter testing according to protocol described below.

Summary of Trial:

Diet and whole body samples were analysed for proximate composition, gross energy (estimated from proximate analysis) and fatty acid composition.

Diet-1 containing the Mbb-fibre complex composition when compared to diet-2 (containing Mbb and fibre separately) displayed an overall improved growth response and survival rate.

Materials & Methods for Shrimp Trial:

Animals
Species: Marsupenaeus japonicas PL15
   Average initial wet animal size 12 mm
Required number: 100×6=1200

Housing
Tanks: 30 L circular aquariums
Number: 6
Volume: 25 liter
Type: Recirculation
Stocking density: 100 animals/tank
Mean Water temperature: 28-29° C.
Lighting: 12 h light, 12 h dark
Watering system: recirculating
Feeding: 3 times per day (8:00, 13:00, 18:00), 1 mm pellets were crumbled
Veterinarian control: Purchased animals are SPF (specific pathogen free)

Data and Analysis
The following parameters were recorded:
   Start: Average weight of the shrimp
   Termination: Average weight of the shrimp
   Start: Average length of the shrimp
   Termination: Average length of the shrimp
   Size variation % CV per group
   Survival: Average survival per group at PL45.

Feeding Trial

Shrimp were fed for 30 days with either diet-1 or diet-2. The different experimental treatments were fed in triplicate groups of shrimp. At the end of the feeding trial, the following biometric parameters were estimated and compared between the dietary treatments:
Final body weight
Final body length
Survival rate

Results:

TABLE 2
Japanese Tiger Prawn growth and survival rates with Mbb-Fiber-
Complex composition supplemented feed

| | | Length at | Length at | Size | | |
|---|---|---|---|---|---|---|
| | Survival | PL-15 | PL-45 | variation | Weight at | Weight at |
| | (%) | (mm) | (mm) | (% CV) | PL-15 (g) | PL-45 (g) |
| Diet 1 | 85 | 12.3 | 22.7 | 8 | 0.3 | 0.6 |
| Diet 2 | 45 | 12.3 | 21.0 | 12 | 0.3 | 0.5 |

Conclusion:

Diet 1, an extruded shrimp feed crumble containing Mbb-Fiber-Complex compositions, prepared according to the invention, demonstrated improved feed performance compared to Diet 2, a control diet where Mbb and fiber had not been prepared such as to form Mbb-Fiber-Complex compositions (in diet 2 probiotics and fiber were added separately). The study supports the hypothesis that integrating probiotics onto the fibrous carrier material matrix of the invention improves the quality of probiotics in extruded feed. The marked increase survival rate of shrimp from Diet 1 group, is also indicative of an improved immune function and/or improved microbiota and/or microbial ecology in the tank water. These in vivo findings corroborate in vitro studies (of example 2) that Probiotic-Fiber-Complexes of the invention afford protection to the Mbb probiotic against the harsh extrusion processing.

### Example 4

1 Introduction

Cotton wool which is a cellulosic fiber material is mainly composed of cellulose (90-95%) and it appears as a very fine, regular fiber with a characteristic twisted structure. Due to its hygroscopic nature, it is very absorbent making it a material ideal for cell colonization, efficient diffusion of oxygen, and transport of nutrients into all parts of the scaffold. Unlike other fibers such as grass or corn pellet that could have been used to simulate the rumen conditions, cotton wool does not fall apart in solution and it provides a neutral backgrounds, indicated especially for spectrophotometric or eventually fluorescence analysis.

The methanogens in the rumen are found free in the rumen fluid, attached to particulate material (fibres), to the rumen epithelium and/or associated as endosymbionts within rumen protozoa and bacteria. Such conditions is simulated by enriching cotton wool with concentrated clarified rumen fluid.

In general, the presence of biofilm is better detected when cells are detached from the fibers.

Ultrasonication is an effective and relatively easy method to disrupt and detach adherent cells/biofilm. Such methods is used to remove biofilm formation from medical implant surfaces that shelters the bacteria and encourages persistence of infection. Ultrasonic energy applied directly to the biomaterial surface is used to disrupt adherent biofilm. Sonication can be used as a method for extraction and measurement of biofilm bacteria. Another method used to quantify biofilm involved the use of the dye crystal violet, which is a basic dye taken up by all bacteria due to its ability to rapidly permeate the cell wall. Such method was adapted from the study of O'Toole et al. (G. A. O'Toole, “Microtiter Dish Biofilm Formation Assay,” J. Vis. Exp., no. 47, pp. 10-11, 2011).

2 Material and Methods

2.1 Cotton Wool Preparation and Rumen Fluid Pre-Treatment

Cotton wool (Naturaline, Coop, Switzerland) and rumen fluid were subject to different treatment as described hereunder:

2.1.1 Autoclavation

Cotton wool (1 g) was soaked into 30 mL or 60 mL of Rumen Fluid, freeze dried (Lyopholizer, Genesis 12 LL, VirTIs, SP Scientific, USA), placed in a serum bottle with 40 mL of medium and autoclaved before inoculation of the cells.

2.1.2 Pasteurization

A stock solution of rumen fluid was heated for 30 min at 55° C. or 65° C. Cotton wool (1 g) was soaked into 30 mL or 60 mL of pasteurized Rumen Fluid, freeze dried (Lyopholizer, Genesis 12 LL, VirTIs, SP Scientific, USA), placed in a serum bottle with 40 mL of medium. Before the inoculum the pasteurization step was repeated.

2.1.3 Sterile Filtration

A stock solution of rumen fluid was sterile filtered (0.22 μm, Steritops, Millipore). Sterile cotton wool (1 g, autoclaved at 121° C. for 20 min) was soaked into 30 mL or 60 mL of sterile filtered rumen fluid and incubated overnight at 37° C. Half of the experiment was carried out by agitating the cotton wool during incubation at 120 rpm and the other without agitation. After the incubation, the cotton wool was freeze dried, placed in serum bottle with 40 mL of pre-sterilised cultivation medium and inoculated.

2.1.4 Combination of Sterile Filtration and Pasteurization

A stock solution of rumen fluid was sterile filtered (0.22 μm, Steritops, Millipore). Sterile cotton wool (1 g, autoclaved at 121° C. for 20 min) was soaked into 30 mL or 60 mL of sterile filtered rumen fluid, pasteurized at 55° C. for 30 min and incubated overnight at 37° C. After the incubation, the cotton wool was freeze dried, placed in serum bottle with 40 mL of pre-sterilised cultivation medium and inoculated.

2.2 Cultivation of the Microorganism

Methanobrevibacter smithii (DSMZ 861) was purchased from the German Collection of Microorganism and Cell Cultures (DSMZ, Braunschweig, Germany) and cultivated in 40 mL of liquid medium (DSMZ medium 119) with 4% (v/V) rumen fluid instead of sludge fluid and fatty acid mixture. The culture was carried out in autoclaved 100 mL serum bottles, sealed with butyl rubber, under sterile anaerobic conditions (1.5 bar, H2/CO2 80%/20%) at 37° C., 120 rpm agitation. Pre-treated cotton wool, enriched with rumen fluid, was added before the inoculum, according to the different experiments. Daily, samples of 0.5 mL were taken and the optical density at 600 nm was measured with a spectrophotometer (Jenway 6320D, Cole Parmer, UK).

2.3 Measurement of Biofilm

Different methods were developed in order to measure the bacteria attached to fibers. The methods were carried out at the end of the growth study, when cells were at the end of the exponential growth phase/beginning stationary phase.

2.3.1 Washing of Cotton Wool

At the end of the cultivation, cotton wool was gently removed with tweezers and a sample of the bacterial suspension remained in the serum bottle was taken and measured at 600 nm. Cotton wool was soaked in 100 mL of PBS and left for 2 minutes. The principle was to remove medium traces and any cell grown in suspension without detaching the biofilm. The washing step was repeated three times in total.

2.3.2 Sonication

After the washing procedure, cotton wool was transferred in a 50 ml test tube with approximately 40 mL of fresh PBS and sonicated in an ultrasonication bath (Emmi-20HC, EMAG German) at maximum power (45 kHz). Samples were taken at time 0, 30 sec, 1 min, 2 min, 3 min, 4 min, 5 min, 10 min and 20 min; optical density was measured at 600 nm.

2.3.3 Crystal Violet Staining

Washed cotton wool was soaked in 100 mL of 0.10% (v/V) crystal violet solution for 15 min and rinsed 3 times with DI water. Crystal violet was solubilized adding 100 mL of 10% (v/V) acetic acid for 15 minutes. Samples were taken and measured at 550 nm.

2.3.4 Re-Cultivation of Cotton Wool

For this part of the experiment, the washing of cotton wool was carried out under sterile conditions to avoid contaminations. The cotton wool was transferred in a pre-sterilised 100 mL serum bottle containing 40 mL of standard cultivation medium. The bottle was sparged with H2:CO2, overpressurized at 1.5 bar and cultivated at 37° C., 120 rpm. Samples were taken at time 0 and every 24 hours.

3 Results

3.1 Rinsing of Cotton Wool in PBS Buffer

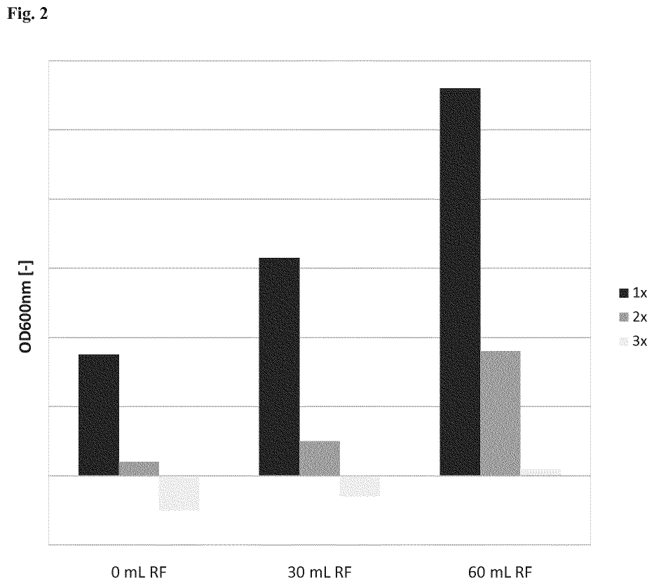

M. smithii was grown until the end of the exponential growth phase (optical density at 600 nm of 2.0) in contact with rumen fluid enriched cotton wool. The next step would be to measure cells attached to the fibers, however an essential step was to first remove traces of medium and of any cell grew in suspension in the medium. In FIG. 2 are represented the results of the cotton wool washing procedure. It was carried out using PBS buffer, and it can be seen that already after the second rinse, the optical density measured at 600 nm drops considerably. After the third rinse, the values are close to 0 for each experiment. Traces of medium and most of the cells growing in suspension were likely to be successfully removed.

3.2 Sonication

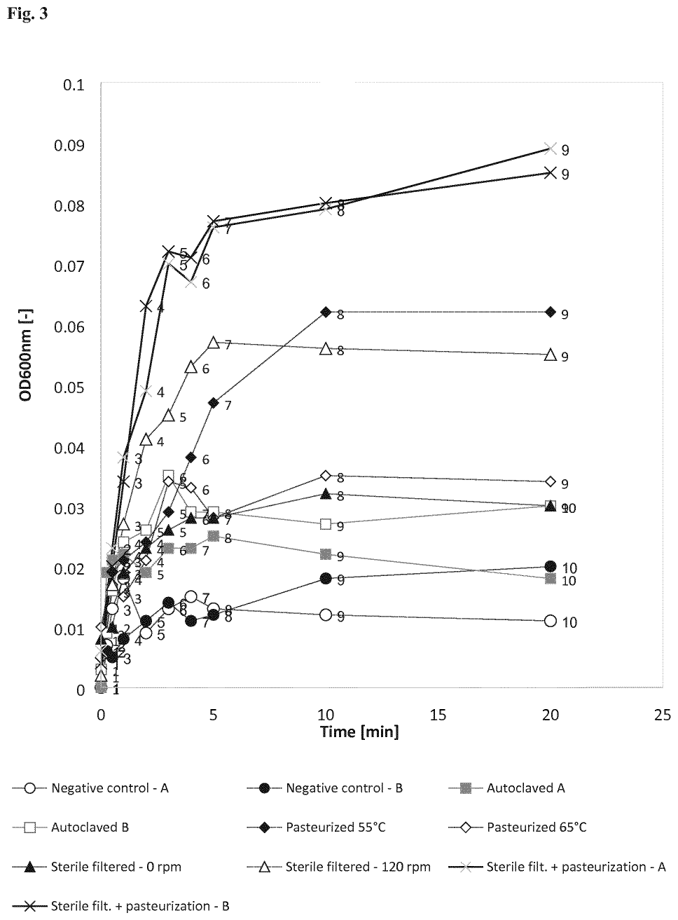

Ultrasounds causes the breakup of biofilm and the dispersion of attached cells. The hypothesis was that when ultrasound are applied on pre-washed cotton wool, the cells detach showing an increase in the absorbance of the solution over the treatment time. In FIG. 3 are shown the results of the study, using freeze dried cotton wool soaked in 60 mL of pre-treated rumen fluid. The negative control was made by adding only sterile cotton wool, without being soaked in rumen fluid. The detailed pre-treatment of rumen fluid is described in Section 2.1.1-2.1.4.

FIG. 3 represents the evolution of optical density at 600 nm of the buffer in which cotton wool is suspended, as a function of the time of ultrasonic treatment. It can be seen that the optical density increases gradually until reaching a plateaux. It suspected that cells attached to cotton wool fibers are detached and released during the treatment. Cotton wool revealed to be a good choice as scaffold, in fact it does not fall apart during the ultrasound treatment compromising the results.

High temperatures and pressure, taking place during autoclavation at 121° C., could affect molecules sensitive to thermal treatment (i.e. protein, antibodies IgA). Results represented in FIG. 3 shows that in both replicate (A, B) when the rumen fluid is autoclaved, the maximum release of cells, in term of OD600 nm was 0.02 and 0.03. Such results are close to the negative control, when cotton wool was not enriched with rumen fluid (0.01 and 0.02).

Milder treatment such as pasteurization at 55° C. and 65° C. were tested, showing a significant increase to 0.06, when rumen fluid was pasteurized at 55° C.

When cotton wool was incubated overnight in sterile filtered rumen fluid under 120 rpm agitation, optical density achieved 0.05 however, only half of the value (0.03) was achieved when during the incubation the cotton wool was not agitated. This suggests that the homogenization allowed a better coating of the fibers with some particular compounds present in the rumen. Consequently, more cells were able to attach.

The best result among all is obtained when the cotton wool is soaked in sterile filtered rumen fluid, followed by a pasteurization step and an overnight incubation at 37° C., under constant agitation (120 rpm). The experiment was repeated in duplicate (A, B) and the maximum OD600 nm was 0.085 and 0.089. This corresponds to an 8-fold increase from the negative control.

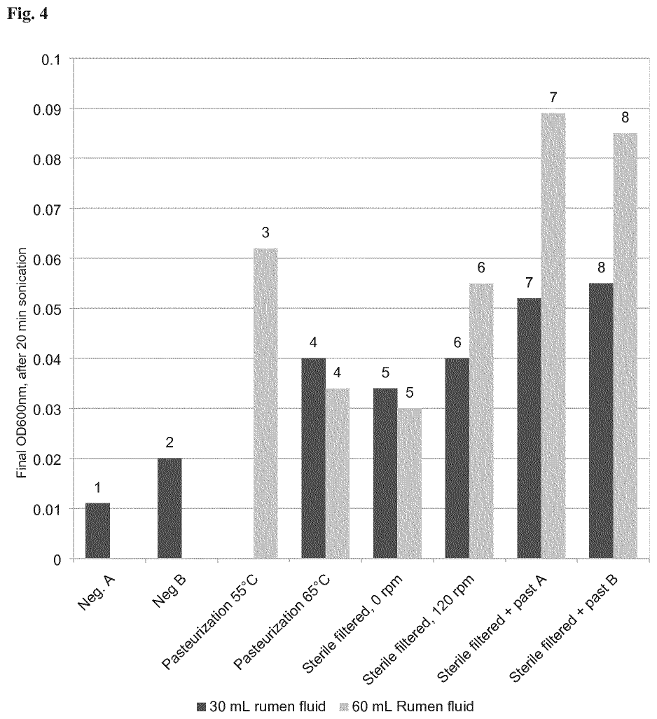

The rationale is that sterile filtration preserves all the proteic compounds or other sensitive molecules present in Rumen Fluid. The difference between the cotton wool/rumen fluid preparation (Section 2.1.3, with agitation at 120 rpm and Section 2.1.4) is only a supplementary pasteurization step before the overnight incubation, however this results in an increase from 0.05 to 0.09. The rationale is that the gentle heating at 55° C. enhances the coating of cotton wool fibers with compounds that are involved in the attachment of cells. When only pasteurization at 55° C. was used as pre-treatment the maximum release was 0.06, showing that the overnight incubation was essential to improve the coating of the fibers. An overview of all the experiments is summarized in FIG. 4. Results of the maximum optical density at 600 nm achieved after 20 min of sonication are compared. It was supposed that the higher the amount of rumen fluid freeze dried with cotton wool, the higher the chances of proteins coating the fibers and therefore enhancing the attachment of M. smithii. Another set of experiments was carried out using half of the volume of Rumen Fluid (30 mL) to soak cotton wool (FIG. 4).

It can be noticed, that results are clearly lower than what obtained when using 60 mL. However, what is interesting is that the results have the same tendency. The highest absorbance was measured when rumen fluid was sterile filtered, pasteurized at 55° C. and incubated overnight under constant agitation.

3.3 Crystal Violet Staining

In order to measure the cells attached to cotton wool fibres, another method was carried out, using crystal violet. The idea was to stain the cotton wool and the potential cells attached, after that the cells are quantified by re-solubilising the dye with acetic acid and measuring it colorimetric at 550 nm. It is assumed that the measure is proportional to the cells attached to cotton wool.

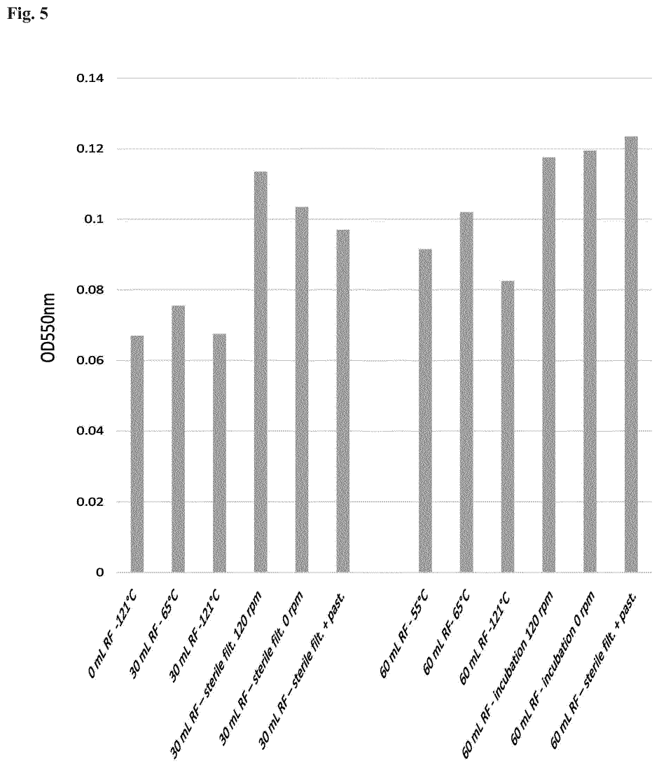

Results showed in FIG. 5 have the same tendency of the results obtained with the sonication. In this case, the maximum release of crystal violet (0.12) was also obtained when cotton wool was sterile filtered, pasteurized and incubate overnight with constant agitation. Such result is 2-fold higher than the negative control (0 mL rumen fluid).

3.4 Re-Growth

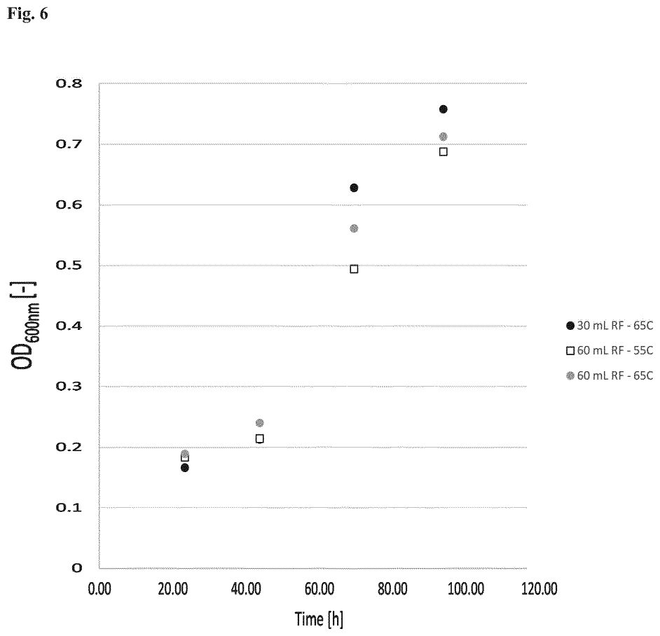

It could be argued that the measurement of optical density after sonication or re-solubilization of crystal violet are not selective to cells. It could be the case that what was measured was the release of rumen fluid proteins attached to the cotton wool instead of adherent cells. Other experiments were carried out: pre-cultivated cotton wool was thoroughly washed in sterile buffer and re-suspended in fresh cultivation medium. As expected, FIG. 6, shows that when pre-cultivated cotton wool is inoculated in fresh medium, the cell growth continues showing an increase in optical density. The morphology of the cell was confirmed under optical microscopy.

Conclusion

The study showed that cotton wool fibers enriched with concentrated clarified rumen fluid can act as a support for the attachment of methanogenic Archaea, in this case of Methanobrevibacter smithii. Rumen fluid was subjected to different conditions to be first of all inactivated, avoiding that other microorganisms would contaminate the growth of M. smithii and second, to enhance the attachment of certain molecules to the cotton wool fibers. The treatment that gave the best results was the sterile filtration of Rumen fluid, followed by a pasteurization treatment at 55° C. and an overnight incubation at 37° C. under constant agitation. The cell attachment was evaluated and described by performing a study with sonication, with crystal violet staining and by re-growing in fresh cultivation medium the cotton wool with the potential cells attached. Results from these analytical methods indicate that cells grow attached to the Rumen Fluid treated fibers and also a large portion of cells continue to grow freely in suspension in the medium.

Compounds present in Rumen Fluid such as immunoglobulin A are involved with the cells attachment. Immunoglobulin A which is present in Rumen Fluid (2 mg/mL) has the capacity of coating the 20% of commensal and pathogenic microbes found in Rumen Fluid.

This particular method to cultivate in vitro M. smithii sets the base for the development of a cultivation system capable to simulate the natural growing conditions found in the gastrointestinal tract of herbivores. The end product will result in fiber complex composition enriched with methanogenic Archaea that can be used as delivery system in various applications (i.e. the probiotic fiber complex composition).

1. A method of manufacturing bioactive prebiotic fibrous material capable of stimulating proliferation of the fiber-associated microbiota, wherein the method is ex-vivo and comprises the steps of:
a) filtering raw rumen content through filters up to 0.20 micron so as to obtain clarified rumen fluid,
b) pasteurizing the clarified rumen fluid of step a) at a temperature of at least 50° C. for at least 1 minute so as to retain heat stable proteins,
c) adding dried cellulosic fiber material to the pasteurized clarified rumen fluid of step b) so as to form a suspension,
d) incubating the suspension at a temperature of 4-41° C. for at least 10 minutes so as to saturate the cellulosic fiber material with the pasteurized clarified rumen fluid, and subsequently collecting the resulting bioactive prebiotic fibrous material.

2. The method of claim 1, wherein a physiologically acceptable cryoprotective reagent is added in step c) and wherein the resulting bioactive prebiotic fibrous material of step d) is freeze-dried so as to form a sterile bioactive prebiotic fibrous material.

3. The method of claim 1, wherein the filtration of step a) is performed by sequential filtering with progressively finer filtration cassettes from 10-0.20 micron filters.

4. The method according to claim 1, wherein the resulting bioactive prebiotic fibrous material comprises Immunoglobulin A (IgA) and/or its secretary components at a concentration of at least 0.1 mg per gram of bioactive prebiotic fibrous material.

5. The method according to claim 2, wherein the physiologically acceptable cryoprotective reagent is selected among the list comprising trihalose, sucrose, ethylene glycol (EG), propylene glycol (PG; 1,2-propanediol), glycerol, formamide, butanediol (BD; 2,3-butanediol) and methylcellulose.

6. The method according to claim 1, wherein the dried cellulosic fiber material of step c) is selected among algae; plants such as forage including lignin, cellulose and structural carbohydrates such as fibrous cellulose and hemi-cellulose.

7. The method according to any claim 1, wherein the weight ratio of the pasteurized clarified rumen fluid to the dried cellulosic fiber material in step c) is 5:1.

8. The method according to claim 1, wherein the incubation of step d) is performed under agitation.

9. A bioactive prebiotic fibrous material capable of stimulating proliferation of the fiber-associated microbiota obtained according to the ex-vivo method of claim 1, wherein the bioactive prebiotic fibrous material comprises rumen factors promoting adhesion and proliferation of cellulolytic-associated microbiota in vitro and in vivo and wherein the rumen factors comprise rumen proteins in the amount of at least 100 mg per liter and wherein Immunoglobulin A and/or its secretary components is present at an amount of at least 0.1 mg per gram of bioactive prebiotic fibrous material, the bioactive prebiotic fibrous material further comprising volatile fatty acids selected among acetic, propionic, valeric and butyric acids.

10. A method of manufacturing probiotic fiber complex composition capable of affording routine industrial feed or food processing comprising thermal stresses selected from −80° C. to at least 120° C. and/or mechanical stresses resulting from extruding, compacting, pelletizing, tableting, compressing or molding where the pressure range is comprised between 0.1 to 700 MPa, the method comprises the steps of:
a) collecting the bioactive prebiotic fibrous material of claim 9,
b) mixing the bioactive prebiotic fibrous material with a fibrolytic microbiota culture in a fermentation culture to form a probiotic integrated material consisting of a probiotic fiber complex composition that is ready for industrial feed or food processing, wherein the fibrolytic microbiota culture comprises at least one population selected from Archaebacteria species.

11. The method of manufacturing probiotic fiber complex material according to claim 9, wherein the fibrolytic microbiota culture of step b) further contains yeast, microbe cells and/or extracts or fragments thereof.

12. The method of manufacturing probiotic fiber complex material of claim 10, wherein the Archaebacteria species is a methanobrevibacter (Mbb).

13. A probiotic fiber complex composition obtained by the method according to claim 10, wherein the probiotic fiber complex composition contains at least 10⁶ live Archaebacteria cells per gram of the probiotic fiber complex composition and at least 0.1 mg of Immunoglobulin A and/or its secretary components per gram of the probiotic fiber complex composition.

14. The probiotic fiber complex composition of claim 13, wherein the probiotic fiber complex composition contains from 10⁶-10¹² live Archaebacteria cells per gram of the probiotic fiber complex composition.

15. A method comprising preparing animal feed with the probiotic fiber complex composition of claim 13.

16. A method comprising preparing animal feed with the bioactive prebiotic fibrous material of claim 9.
